SEQUENCE LISTING (14 sequences)

SEQ ID NO: 1 - protein, 173 aa, Hepatitis B virus
GGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTN

SEQ ID NO: 2 - protein, 173 aa, Hepatitis B virus
GGWSSKPRKGMGTNLSVPNPLGFFPDHQLDPAFKANSDNPDWDLNPHKDNWPDSNKVGVGAFGLGFTPPHGGLLGWSPQAQGILTTVPAAPPPASTNRQVARPPTPLSPPLRDTHPQAMQWNSTTFHQTLQDPRVRALYFPAGGSSSGTVNPVQNTASSISSILSKTGDPVPN

SEQ ID NO: 3 - protein, 173 aa, Hepatitis B virus
GGYSSKPRKGMGTNLSVPNPLGFLPDHQLDPAFGANSNNPDWDFNPNKDPWPEAWQVGVGAFGPGFTPPHGGLLGWSPQAQGILTPVPAAPPPASTNRQSGRQPTPISPPLRDSHPQAMQWNSTTFHQALLDPRVRGLYFPAGGSSSGTTNPVPTTASPISSIFSRTGDPAPN

SEQ ID NO: 4 - protein, 162 aa, Hepatitis B virus
GQNLSTSNPLGFFPDHQLDPAFRANTANPDWDFNPNKDTWPDANKVGAGAFGLGFTPPHGGLLGWSPQAQGILQTLPTNPPPASTNRQSGRQPTPLSPPLRNTHPQAMQWNSTTFHQTLQDPRVRGLYFPAGGSSSGTVNPVPTTVSHISSIFSRIGDPALN

SEQ ID NO: 5 - protein, 172 aa, Hepatitis B virus
GLSWTVPLEWGKNHSTTNPLGFFPDHQLDPAFRANTRNPDWDHNPNKDHWTAANKVGVGAFGPGFTPPHGGLLGWSPQAQGMLKTLPADPPPASTNRQSGRQPTPITPPLRDTHPQAMQWNSTTFHQALQDPRVRGLYFPAGGSSSGTVNPVPTTASLISSIFSRIGDPAPN

SEQ ID NO: 6 - protein, 173 aa, Hepatitis B virus
GAPLSTTRRGMGQNLSVPNPLGFLPDHQLDPLFRANSSSPDWDFNKSKDNWPMANKVGVGGYGPGFTPPHGGLLGWSPQAQGVLTTLPADPPPASTNRRSGRKPTPVSPPLRDTHPQAMQWNSTQFHQALLDPRVRALYFPAGGSSSETQNPAPTIASLTSSIFLKTGGPAMN

SEQ ID NO: 7 - protein, 172 aa, Hepatitis B virus
GLSWTVPLEWGKNLSTSNPLGFLPDHQLDPAFRANTNNPDWDFNPKKDPWPEANKVGVGAYGPGFTPPHGGLLGWSPQSQGTLTTLPADPPPASTNRQSGRQPTPISPPLRDSHPQAMQWNSTAFHQALQNPKVRGLYFPAGGSSSGIVNPVPTIASHISSIFSRIGDPAPN

SEQ ID NO: 8 - protein, 173 aa, Hepatitis B virus
GAPLSTARRGMGQNLSVPNPLGFFPDHQLDPLFRANSSSPDWDFNTNKDNWPMANKVGVGGFGPGFTPPHGGLLGWSPQAQGILTTSPPDPPPASTNRRSGRKPTPVSPPLRDTHPQAMQWNSTQFHQALLDPRVRGLYFPAGGSSSETQNPAPTIASLTSSIFSKTGDPAMN

SEQ ID NO: 9 - protein, 30 aa, synthetic construct
EAAFNDAIKASTGGAYESYKFIPALEAAVK

SEQ ID NO: 10 - protein, 37 aa, synthetic construct
AEEVKVIPAGELQVIEKVDAAFKVAATAANAAPANDK

SEQ ID NO: 11 - protein, 33 aa, synthetic construct
ADLGYGPATPAAPAAGYTPATPAAPAEAAPAGK

SEQ ID NO: 12 - protein, 33 aa, synthetic construct
ATTEEQKLIEKINAGFKAALAAAAGVQPADKYR

SEQ ID NO: 13 - protein, 307 aa, synthetic construct
MEAAFNDAIKASTGGAYESYKFIPALEAAVKAEEVKVIPAGELQVIEKVDAAFKVAATAANAAPANDKGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNADLGYGPATPAAPAAGYTPATPAAPAEAAPAGKATTEEQKLIEKINAGFKAALAAAAGVQPADKYR

SEQ ID NO: 14 - protein, 306 aa, synthetic construct
EAAFNDAIKASTGGAYESYKFIPALEAAVKAEEVKVIPAGELQVIEKVDAAFKVAATAANAAPANDKGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNADLGYGPATPAAPAAGYTPATPAAPAEAAPAGKATTEEQKLIEKINAGFKAALAAAAGVQPADKYR

(19) United States
(12) Patent Application Publication
(10) Pub. No.: US 2024/0382584 A1
(43) Pub. Date: Nov. 21, 2024

(54) HBV VACCINE INDUCING PRES-SPECIFIC NEUTRALIZING ANTIBODIES

(71) Applicant: Viravaxx AG, Wien (AT)

(72) Inventors: Inna TULAEVA, Wien (AT); Carolin CORNELIUS-NIKL, Wien (AT); Pia GATTINGER, Vienna (AT); Rudolf VALENTA, Theresienfeld (AT)

(73) Assignee: Viravaxx AG, Wien (AT)

(21) Appl. No.: 18/693,176

(22) PCT Filed: Sep. 23, 2022
(86) PCT No.: PCT/EP2022/076518

§ 371 Date: Mar. 19, 2024

(30) Foreign Application Priority Data
Sep. 23, 2021 (EP) 21198517.1

(51) Int. Cl. A61K 39/29; A61K 39/00; A61K 39/36; A61P 31/20; C12N 7/00

(52) U.S. Cl. CPC A61K 39/292; A61K 39/36; A61P 31/20; C12N 7/00; A61K 2039/545; A61K 2039/55505; A61K 2039/55561; A61K 2039/55566; A61K 2039/55572; A61K 2039/575; C12N 2730/10122; C12N 2730/10134

(57) ABSTRACT

A Hepatitis B virus (HBV) vaccine comprising a fusion protein of a preS polypeptide fused to at least one grass pollen allergen peptide, for use in the treatment of a subject to induce HBV neutralizing antibodies, wherein the subject is an immune tolerant human subject and said treatment comprises repeated vaccination to break immune tolerance against HBV.

### FIELD OF THE INVENTION

The present invention relates to novel treatment of immune tolerant subjects to induce an anti-HBV immune response. The present invention refers to the new medical use of a vaccine capable of inducing preS-specific HBV-neutralizing antibodies in chronically HBV-infected patients and non-responders to HbSAg-based vaccines.

### BACKGROUND OF THE INVENTION

Hepatitis B virus (HBV) is a small double shelled virus in the hepadnaviridae family. Humans are the only known host for this virus. HBV is a serious threat and contributes significantly to healthcare cost worldwide. According to the U.S. Centers for Disease Control (CDC), an estimated 2 billion persons worldwide have been infected with HBV, and more than 350 million persons have chronic, lifelong infections. HBV infection is an established cause of acute and chronic hepatitis and cirrhosis. It is responsible for up to 80% of hepatocellular carcinomas. The World Health Organization estimated that approx. 900,000 persons died worldwide in 2015 of hepatitis B-associated acute and chronic liver disease.

While most acute HBV infections in adults result in complete recovery, fulminant hepatitis occurs in about 1% to 2% of acutely infected persons. About 200 to 300 Americans die of fulminant disease each year (case-fatality rate 63% to 93%). Although the consequences of acute HBV infection can be severe, most of the serious complications associated with HBV infection are due to chronic infection.

Chronic infection is responsible for most of the HBV-related morbidity and mortality, including chronic hepatitis, cirrhosis, liver failure, and hepatocellular carcinoma. Approximately 25% of persons with chronic HBV infection die prematurely from cirrhosis or liver cancer. Chronic active hepatitis develops in more than 25% of carriers and often results in cirrhosis. An estimated 3,000 to 4,000 persons die of hepatitis B-related cirrhosis each year in the United States. Persons with chronic HBV infection are at 12 to 300-times higher risk of hepatocellular carcinoma than non-carriers, leading to an estimated death rate of 1,000 to 1,500 persons each year in the United States from hepatitis B-related liver cancer.

Approximately 5% of all acute HBV infections progress to chronic infection, with the risk of chronic HBV infection decreasing with age. As many as 90% of infants, who acquire HBV infection from their mothers at birth become chronically infected. Of children who become infected with HBV between 1 year and 5 years of age, 30% to 50% become chronically infected.

The CDC and WHO recommend HBV vaccination now in all new born infants as well as for persons living at risk of acquiring an infection (e.g., healthcare workers, travellers to endemic regions or diabetics). Immunization campaigns have been implemented in many countries and have led to a marked reduction in new infections. The proportion of children under 5 years of age who become chronically infected fell from 4.7% to 1.3%.

While vaccination with established vaccines based on the S-protein (i.e., HbSAg) is safe and well tolerated, approx. 15% of persons treated with them fail to elicit significant titres of anti-HBV antibodies. Seroconversion rate is highest in early childhood (>95%) and decreases significantly with age (<65% at age 50 and above) and in a number or risk groups like patients with immune deficiencies or on dialysis. Therefore, there is a medical need to develop an improved hepatitis B vaccine at least for non-responders to conventional immunization with HbSAg-based vaccines.

The use of established HbSAg-based HBV vaccines as well as the prolonged treatment with nucleotide/nucleoside drugs has proven futile to establish a functional cure of chronically infected patients. After withdrawal of these drugs, in many cases a recurrence of disease is observed.

This is probably due to the unique lifecycle of the virus, which leads to the incorporation of multiple copies of the viral genome in circular form in the nucleus of infected cells. This so called cccDNA effects a constant production of new virions and a permanent stimulation of the immune system. This chronic inflammation is the core reason for the development of cirrhosis and liver carcinoma. A functional cure of the disease is therefore only possible if the infected liver can get cleared from hepatocytes harboring cccDNA. Multiple pathways for this clearance are conceptually feasible.

HBV vaccines based on HBsAg and manufactured in yeast cells have been shown to be non-effective for therapeutic vaccination. Chronically HBV-infected patients mount low or no antibodies against the preS protein (Immunotherapy With the preS-based Grass Pollen Allergy Vaccine BM32 Induces Antibody Responses Protecting Against Hepatitis B Infection. Cornelius C, Schöneweis K, Georgi F, Weber M, Niederberger V, Zieglmayer P, Niespodziana K, Trauner M, Hofer H, Urban S, Valenta R. EBioMedicine. 2016 September; 11:58-67. doi:10.1016/j.ebiom.2016.07.023. Epub 2016 Aug. 8; EP3138579A1) which contains the binding site for the sodium taurocholate co-transporting polypeptide (NTCP) receptor for HBV which would protect against chronic reinfection of liver cells. Thus, they seem to be immune-tolerant against preS. Furthermore, they produce abundantly sub-viral particles presenting HBsAg which may adsorb S-specific antibodies. Thus, the S-specific antibodies cannot always prevent infection of liver cells.

EP3138579A1 and WO 2017/037280 disclose a fusion protein comprising a hepatitis B preS polypeptide and respective vaccine for use in the treatment and/or prevention of a hepatitis B virus. In this document it has been shown that vaccination of HBV non-infect subjects and HBV naïve rabbits can induce preS-specific antibodies. However, EP3138579A1 and WO 2017/037280 did not show that it is possible to increase/induce preS-specific HBV neutralizing antibodies in patients who are chronically infected with HBV. Neither EP3138579A1 nor WO 2017/037280 provide any evidence that it is possible to induce in non-responders to S-protein-based HBV vaccines a preS-specific immune response which can neutralize HBV infections.

A respective vaccine antigen is a fusion of peptides derived from four timothy grass pollen allergens (Phl p 1, 2, 5 and 6) fused to the N- and C-terminus of the preS domain of the Hepatitis B virus (HBV) surface antigen (Focke-Tejkl et al. J. Allergy Clin. Immunol. 2015; 135:1207-1217), and expressed as fusion proteins in Escherichia coli. The vaccine not only induces allergen-specific IgG antibodies, but in addition also high titers of preS specific antibodies. The documents do not demonstrate that the polypeptide can induce neutralizing antibodies in chronically HBV infected patients or non-responders to S-protein-based HBV vaccines (Cornelius C. et al. EBioMedicine. 2016; 11:58-67).

WO2012/168487A1 discloses a fusion protein comprising a hepatitis B preS polypeptide fused to allergen-derived peptides as allergy vaccine but does not show any evidence that the allergy vaccines can induce HBV-specific neutralizing immune responses.

A vaccine under development, designated VVX001 (Viravaxx AG, Vienna, Austria) comprises as a vaccine antigen a fusion protein of preS with epitope peptides from Phl p 5.

There is a need for effective vaccine treatment inducing immunity against HBV in subjects to develop neutralizing antibodies in chronically HBV-infected patients and in non-responders to S-protein based HBV vaccines.

### SUMMARY OF THE INVENTION

It is the objective of the present invention to provide new treatment options for prophylaxis or therapy against HBV infection in human subjects, in particular in subjects with immunological tolerance such as in chronically HBV-infected patients and in non-responders to S-protein based HBV vaccines who are accordingly immune tolerant to certain HBV antigens.

This objective is solved by the subject claimed and as further described herein, in particular by the present examples.

In the current invention, antibody responses and an immune response obtained by immunization with a HBV vaccine based on a preS polypeptide fused to at least one grass pollen allergen peptide were achieved, even in chronically HBV-infected patients and in non-responders to S-protein based HBV vaccines. It could be shown that repeated vaccination of the subjects with the described preS-based vaccine unexpectedly could induce in chronically HBV-infected patients and in non-responders to S-protein based HBV vaccines, the production of preS-specific antibody and T cells responses and that the preS-specific antibodies were able to neutralize HBV infections.

Immune tolerance, or immunological tolerance, or immunotolerance (herein also referred to as “immune tolerance”) is commonly understood as a state of unresponsiveness of the immune system so that no adaptive (i.e., antibody, T cell responses) are mounted to certain antigens, substances or tissues. Chronically HBV-infected patients and non-responders to S-protein based HBV vaccines are herein understood as being immune tolerant subjects, because not developing a sufficient antibody and/or T cell response upon exposure to respective HBV antigen(s).

Therefore, subjects who are immunologically tolerant were successfully immunized according to the invention, which is herein understood as “breaking immunological tolerance”.

Therefore, the vaccine described herein can be effectively used in a large population, including chronically HBV-infected patients and non-responders to S-protein based HBV vaccines.

It has been shown that a preS1 peptide-based competitive inhibitor termed Myrcludex B (Bulevirtide) containing an epitope targeted by antibodies induced by a PreS vaccine such as described herein can block entry of HBV and also hepatitis D virus (HDV). Therefore, the subject vaccine and treatment described herein not only refers to HBV, but also to HDV. (Blocking Entry of Hepatitis B and D Viruses to Hepatocytes as a Novel Immunotherapy for Treating Chronic Infections. Maravelia P, Frelin L, Ni Y, Caro Pérez N, Ahlén G, Jagya N, Verch G, Verhoye L, Pater L, Johansson M, Pasetto A, Meuleman P, Urban S, Sällberg M. J Infect Dis. 2021 Jan. 4; 223(1):128-138. doi:10.1093/infdis/jiaa036). Therefore the preS-based vaccine described herein will also be effective against HDV infections.

Thus, the invention provides for a Hepatitis B virus (HBV) vaccine comprising a fusion protein of a preS polypeptide fused to at least one grass pollen allergen peptide (herein also referred to as “preS fusion protein”), for use in the treatment of a subject to induce HBV neutralizing antibodies, wherein the subject is an immune tolerant subject such as a subject that is a chronically HBV-infected patient or a non-responder to S-protein based HBV vaccines. Specifically, said treatment comprises repeated vaccination to break immune tolerance against HBV.

Specifically, the treatment induces HBV and HDV neutralizing antibodies.

According to a specific aspect, the medical use described herein provides for a respective method of treatment of a subject in need thereof, such as for therapy or prevention of a disease, in particular a disease caused by HBV, or HBV and HDV, or HDV, such as hepatitis or chronic hepatitis.

Specifically, the medical use involves an immunotherapy, such as an active immunotherapy by vaccination and immunizing a subject in need thereof. Specific immunotherapies provide for the treatment of a subject afflicted with, or at risk of contracting or suffering a disease or recurrence of a HBV and/or HDV disease, by a method comprising inducing, enhancing, suppressing or otherwise modifying a HBV-specific immune response.

According to a specific aspect, the subject is a human subject.

Specifically, the human subject is:
a) a patient with an acute or chronic HBV infection
b) a patient who is chronically infected with HBV, optionally wherein said patient is of an inactive carrier status; and/or
c) a low- or non-responder to a HBV vaccine, such as a HBsAg-based vaccine, or an S-protein-based HBV vaccine, or to such HBV vaccine that does not comprise said preS fusion protein.

According to a specific aspect, the subject is on standard antiviral treatment, preferably with nucleos(t)ide (NUC) treatment, or wherein the subject has discontinued standard antiviral treatment, such as NUC treatment, e.g., 2 weeks to 24 weeks prior to receiving the vaccine described herein.

Specifically, the human subject is treated within a larger population of treated subjects, such as to include both, subjects who have a HBV infection, and subjects for whom it is not known if there will be a response to S-protein based vaccines or if there are non-responders to S-protein-based HBV vaccines.

Specifically, the subject is a HBsAg positive patient and/or at risk of suffering from HBV and/or HDV disease or recurrent disease.

Specifically, the patient is chronically infected with HBV, and/or is at risk of suffering from HBV disease or recurrent HBV disease, and/or chronically infected with HDV, and/or is at risk of suffering from HDV disease or recurrent HDV disease. Specifically, the medical use described herein provides for the respective therapeutic treatment against such hepatitis virus disease, in particular a HBV and/or HDV disease, or recurrent disease.

A hepatitis B vaccine “non-responder” refers to a person who does not develop protective surface antibodies after completing two full series of the hepatitis B vaccine and for whom an acute or chronic hepatitis B infection has been ruled out. Although the majority of persons vaccinated against hepatitis B successfully respond to vaccination, an estimated 5-15% of persons may not respond due to older age, obesity, smoking, and other chronic illness.

Specifically, a non-responder to a HBV vaccine comprises less than 10 IU/L anti-HBs antibodies in serum.

Specifically, a low-responder to a HBV vaccine comprises less than 100 IU/L anti-HBs antibodies in serum.

Specifically, a low- or non-responder to a conventional HBV vaccine such as e.g., a HBsAg based vaccine, is at risk of being infected with HBV upon exposure to the virus. Specifically, the medical use described herein provides for the respective prophylactic treatment against such HBV infection and a respective HBV disease or recurrent disease.

Specifically, the HBV disease and/or HDV disease is hepatitis, such as e.g., acute, chronic or recurrent hepatitis.

According to a specific aspect, the subject is diagnosed with chronic hepatitis B-infection, with or without active hepatitis. Specifically, the subject is a carrier of inactive HBV or hepatitis.

Specifically, the subject is diagnosed with chronic hepatitis B-infection without active hepatitis, and
a) comprises a level of HBsAg of less than 3000 IU/ml blood or which is HBsAg negative before vaccination; and/or
b) comprises a HBV DNA viral load of less than 2000 IU/ml blood, or wherein HBV DNA is not detectable.

According to a specific aspect, said treatment is a prophylactic or therapeutic treatment, preferably for the therapy of chronic hepatitis B-infection and/or chronic hepatitis D-infection.

The invention further provides for a vaccine comprising the vaccine antigen described herein in an effective amount, such as an immunogenic effective amount.

Specifically, the vaccine comprises an effective amount of the vaccine antigen e.g., ranging between 0.001-1 milligram per dose, preferably between 10-100 or 15-50 microgram preS or preS fusion protein, such as about 20, 40, 60 or 80 microgram preS or preS fusion protein per dose.

Specifically, the vaccine antigen described herein is provided in a pharmaceutical preparation comprising a pharmaceutically acceptable carrier e.g., to provide an immunogenic formulation or vaccine.

Specifically, the vaccine is administered to the subject by subcutaneous, intramuscular, intranasal, microneedle, mucosal, skin, or transdermal administration. A respective formulation may be used, for use in the subcutaneous, intramuscular, intranasal, microneedle, mucosal, skin, or transdermal administration.

The amount of vaccine antigen that may be combined with excipients to produce a single dosage form will vary depending upon the particular mode of administration. The dose of the vaccine antigen may vary according to factors such as age, sex and weight of the subject, and the ability to elicit the desired antibody response in the subject.

Dosage regime may be adjusted to provide the optimum therapeutic response. For example, several divided doses may be administered daily or the dose may be proportionally reduced as indicated by the exigencies of the therapeutic situation. The dose of the vaccine may also be varied to provide optimum preventative dose response depending upon the circumstances.

Specifically, the vaccine described herein can be administered to the subject in an effective amount employing a prime-boost strategy.

For instance, the vaccine described herein may be administered to a subject several times according to a prime-boost regiment, at time intervals between the subsequent vaccinations ranging between 2 weeks and 5 years, preferably between 1 month and up to 3 years, more preferably between 2 months and 1.5 years. Specifically, the vaccine described herein is administered between 2 and 10, preferably between 2 and 7, even more preferably up to 5 and most preferably up to 3 times.

Specifically, repeated vaccination comprises:
a) a basic immunization comprising at least 2, 3, 4, 5, preferably up to 5, injections at intervals of 4-8 weeks, or in monthly intervals, and
b) booster injections of at least 2, 3, 4, preferably up to 4 injections per year, following basic immunization.

Typically, a first booster injection starts after at least one, two or three months following the basic immunization.

According to a specific embodiment, two or three doses are administered at time intervals of 3-6 weeks, such as e.g., once a month or at intervals of 3-4 weeks, or intervals between 4-8 weeks or monthly intervals, to establish an immune response. The immune response can be boosted by administering one or more further doses e.g., at intervals of about 1, 2, 3, 4, 5, 6, 7, 8, 9, 10, 11, or 12 months. According to a specific regimen, 2, 3, 4, 5, or 6 prime doses are administered, before at least 1, 2, 3, 4, 5, or 6 booster doses are administered, as needed to keep the antibody levels high.

According to a specific aspect, said treatment comprises repeated vaccination (e.g., subcutaneous injections) administering at least 3, 4, 5, 6, 7, 8, 9, 10 doses, or even more frequently, as appropriate to control viral load in HBV infected patients.

Specifically, repeated vaccination comprises at least three maintenance doses followed by one, two or more booster doses.

Specifically, repeated vaccination is done at intervals of at least 3, 4, 5, 6, 7, 8, 9, 10, 11, or 12 weeks, such as e.g., at intervals of 4-8 weeks, or in monthly intervals.

According to a specific aspect, said treatment comprises repeatedly administering the vaccine by subcutaneous injections, in particular by at least 3, 4, 5, 6, 7, 8, 9, 10 injections in monthly intervals, or at intervals of about 4 weeks (+/−5 days).

According to a specific aspect, the preS polypeptide comprises at least 50% length of any one of SEQ ID NO:1-8, and at least 80% sequence identity to the corresponding region of the respective SEQ ID NO: 1-8, preferably wherein the preS polypeptide comprises of consists of SEQ ID NO:1.

According to specific examples, a preS polypeptide is used in the fusion protein which comprises or consists of at least any one of 80%, 85%, 90%, 95%, or 100% sequence identity to a viral protein, preferably selected from the group consisting of:
a) any one of a Hepatitis B preS protein or fragment thereof, such as a polypeptide comprising or consisting of any one of SEQ ID NO: 1-8; or
b) a derivative or mutant of any of the foregoing (the parent sequence), which comprises at least 50% (or at least any one of 60%, 70%, 80%, 85%, 90%, 95%, 96%, 97%, 98%, 99%, or 100%) of the length of the parent sequence and at least 80% (or at least any one of 85%, 90%, 95%, 96%, 97%, 98%, 99%, or 100%) sequence identity to the parent sequence, which may or may not be an artificial mutant comprising one or more point mutations which point mutation(s) may characterize one or more natural virus mutants, or may evolve through mutagenesis by a directed evolution approach mutagenizing a parent virus sequence.

Specific HBV preS polypeptides may be used, such as comprising or consisting of a polypeptide comprising at least any one of 80%, 85%, 90%, 95%, or 100% sequence identity to the natural preS protein or one or more fragments thereof. Specific HBV preS polypeptides may originate from (or derived from) any one of the HBV genotypes B, C, D, E, F, G or H, or a subtype thereof. Subtypes of hepatitis B viruses include A1, A2, A3, A4, A5, B1, B2, B3, B4, B5, C1, C2, C3, C4, C5, D1, D2, D3, D4, D5, F1, F2, F3 and F4 as discussed in Schaefer et al. (World J Gastroenterol. 2007; 13:14-21).

One or more preS polypeptides may be comprised in the fusion protein. The presence of more than one hepatitis B preS polypeptides in the fusion protein may have the advantage that more antigens are presented to the immune system allowing the formation of antibodies directed to preS. HBV preS polypeptides being part of the fusion protein of the present disclosure may be derived from the same HBV genotype or from different genotypes. For example, the fusion protein described herein may comprise the preS polypeptide of HBV genotype A, B, C, D, E, F, G or H, or a subtype thereof only, or may be combined with a further (the same or a different) preS polypeptide derived from any one of the (same or different) HBV genotypes A, B, C, D, E, F, G or H, or a subtype thereof.

Fragments of a preS protein suitably used as heterologous element in the fusion protein consist preferably of at least any one of 30, 40, or 50 consecutive amino acid residues of the preS protein sequence, preferably between aa1-70 of the hepatitis B preS protein consisting of any one of SEQ ID NO:1-8, whereby SEQ ID NO:1-8 belong to HBV genotypes B to H, respectively. Specific fragments may comprise preS1 and/or preS2 of the hepatitis B preS protein. Using preS as a heterologous protein in a vaccine as described herein, induces antibodies that prevent HBV infection (Cornelius C. et al. EBioMedicine. 2016; 11:58-67).

According to a specific aspect, the grass pollen allergen peptides are non-allergenic peptides from the IgE binding sites of any one or more of the four major grass pollen allergens, Phl p 1, Phl p 2, Phl p 5 and Phl p 6, or any isoallergen or naturally-occurring variants thereof. Recombinant isoallergens, fragments, mutants or synthetic peptides of grass pollen allergen are preferably used, which are not allergenic as determined in a standard in vitro, ex vivo or in vivo assay.

Specifically, the grass pollen allergen peptides are isolated peptides originating from a natural source, or artificial peptides comprising a certain sequence identity thereto.

Specifically, the number of grass pollen allergen peptides comprised in the fusion protein is at least one, two, three or four, which may be different from each other, or may include a number of peptides, such as e.g., 1, 2, 3, or 4 peptides, which are the same or having any one of 80%, 85%, 90%, 95%, or 100% sequence identity to each other.

Any of the grass pollen allergen peptides may be comprised in the fusion protein only once, or be repeatedly comprised in the fusion protein, such that at least two, three or four grass pollen allergen peptides with at least any one of 80%, 85%, 90%, 95%, or 100% sequence identity are comprised in one fusion protein.

Specifically, at least one, two, three or four of the grass pollen allergen peptides are of the grass pollen allergen Phl p 5, such as e.g., of the source: Plantae Liliopsida, Order: Poales, Species: Phleum pratense (Timothy), or any isoallergen or naturally-occurring variants thereof. Phl p 5 is a 29 kDa major allergen from timothy grass pollen, and one of the most reactive members of group 5 allergens. Its sequence comprises two repeats of a novel alanine-rich motif.

Phl p 5 peptides are preferably used in the vaccine described herein, such as e.g., one or more, or all of the following: SEQ ID NO:9, 10, 11, or 12, or the respective peptide(s) comprising or consisting of an amino acid sequence having at least any one of 80%, 85%, 90%, 95%, or 100% sequence identity of any of the foregoing.

According to specific examples, said grass pollen allergen peptide comprises or consists of an amino acid sequence having at least any one of 80%, 85%, 90%, 95%, or 100% sequence identity to the natural grass pollen allergen peptide. Specific examples refer to the natural grass pollen allergen peptide comprising or consisting of any one of SEQ ID NO:9, SEQ ID NO: 10, SEQ ID NO: 11 or SEQ ID NO:12, or an amino acid sequence having at least any one of 80%, 85%, 90%, 95%, or 100% sequence identity to any one of SEQ ID NO:9, SEQ ID NO:10, SEQ ID NO:11 or SEQ ID NO:12.

According to a specific aspect, the grass pollen allergen peptide comprises or consists of any one of, or a fusion of more than one of SEQ ID NO:9-12.

Specifically, any one, two three or four of the peptides comprising or consisting of at least 80%, 85%, 90%, 95%, or 100% sequence identity to SEQ ID NO:9, 10, 11, or 12 may be used in the fusion protein. The fusion of at least two peptides may be used as a tandem fusion, such as a sequential fusion (with or without a linking sequence), and/or with overlapping parts, e.g., peptide sequences that overlap in 1, 2, 3, 4, 5, 6, 7, or 8 amino acids at one or both, the N-terminal end and the C-terminal end.

According to a specific aspect, the fusion protein is a single-chain fusion protein, wherein preS polypeptide and said at least one grass pollen allergen peptide are positioned in any order.

Specifically, at least one or two grass pollen allergen peptides are fused to the N-terminus and/or at least one or two grass pollen allergen peptides are fused to the C-terminus of the preS polypeptide, wherein said construct may comprise the grass pollen allergen peptides of the same type or sequence, or different ones.

An exemplary fusion protein comprises at least two grass pollen allergen peptides at the N-terminal end of the preS polypeptide, and at least two of the grass pollen allergen peptides at the C-terminal end of the preS polypeptide.

According to a specific embodiment, the fusion protein comprises or consists of an amino acid sequence having at least any one of 80%, 85%, 90%, 95%, or 100% sequence identity to SEQ ID NO:13 or SEQ ID NO:14.

According to a specific example, the grass pollen allergen peptides SEQ ID NO:9, 10, 11, and 12 are fused to a HBV preS amino acid sequence e.g., any one of SEQ ID NO: 1-8, to provide a vaccine antigen.

For example, SEQ ID NO:13 comprises a fusion of
a) the grass pollen allergen peptides SEQ ID NO:9, 10, 11, and 12, wherein
   i) a first peptide fusion consists of a direct fusion of SEQ ID NO:9 and SEQ ID NO: 10, such that the C-terminus of SEQ ID NO:9 is directly fused to the N-terminus of SEQ ID NO:10; and
   ii) a second peptide fusion consists of a direct fusion of SEQ ID NO:11 and SEQ ID NO:12, such that the C-terminus of SEQ ID NO: 11 is directly fused to the N-terminus of SEQ ID NO:12; and
b) a preS sequence consisting of SEQ ID NO:1,
wherein the N-terminus of the preS sequence is fused to the C-terminus of the first peptide fusion, and the C-terminus of the preS sequence is fused to the N-terminus of the second peptide fusion.

Alternative constructs comprising two peptides fused in tandem at the N-terminus of the preS sequence, and two peptides fused in tandem at the C-terminus of the preS sequence may be produced.

The fusion protein or a tandem fusion may or may not comprise one or more linking sequences, such as to link any of the peptides or polypeptide. Specifically, the linking sequence can be a peptide or polypeptide or protein domain.

Specifically, the linker can be a linker of varying length, such as a peptide, polypeptide or protein domain linker (also referred to as peptidic linker). The length of the linker is variable, typically ranging between 5 and 15 amino acids. Longer linkers can be used e.g., when necessary to ensure that two adjacent elements do not sterically interfere with each other, or when introducing a heterologous peptide, polypeptide or protein domain.

Linkers can be composed of flexible residues like glycine and serine so that the adjacent peptides are free to move relative to one another. Exemplary peptidic linker comprise or consist of a sequence of a number of G and/or S.

According to specific examples, a linker may be used that is commonly used in a single chain variable fragment (Fv) antibody construct comprising a variable heavy (VH) domain linked to a variable light (VL) domain.

According to a specific aspect, the vaccine antigen may comprise one or more peptidic spacers in addition to a linker, such as to improve the structure or stability of the polypeptide. Specifically, the spacer can be a peptide or polypeptide or protein domain.

Yet, the fusion protein described herein may comprise the elements to be fused which can be bound to each other by bioconjugation, chemical conjugation or cross-linking. For example, the vaccine antigen may comprise multimerization domains, carriers, or devices such as nanostructures or beads that are suitably used to immobilize a series of polypeptides.

SEQ ID NO:13 comprises no heterologous linker or spacer sequence.

The fusion protein may or may not comprise an N-terminal and/or C-terminal extension, such as an N-terminal methionine (“M”) as included in SEQ ID NO:13, extending the N-terminus of the first peptide fusion. Specifically, a construct can be provided with or without any N-terminal and/or C-terminal extension. Specifically, the extension can be a peptide or polypeptide or protein domain.

Specifically, the vaccine antigen may consist of SEQ ID NO:13, or the sequence of SEQ ID NO:13 without the N-terminal “M”; such as depicted in SEQ ID NO:14.

According to a specific aspect, the vaccine antigen can be provided as a nucleic acid molecule that encodes the preS fusion protein described herein, preferably comprising a polynucleotide sequence comprising at least 60%, 70%, 80%, 90%, 95%, 96%, 97%, 98%, 99%, or 100% sequence identity to a nucleotide sequence encoding any of the preS fusion proteins described herein. A coding nucleic acid molecule, such as a cDNA, can be used for producing the vaccine antigen in vitro. A coding nucleic acid molecule, such as an RNA, can be used to produce an RNA-vaccine.

Exemplary polynucleotide sequences are codon-optimized sequences, which are optimized for recombinant expression in the respective host cell or for expression in the subject e.g., a human being. Specific exemplary polynucleotide sequences are mRNA sequences.

Specific embodiments of a vaccine comprise a nucleic acid molecule encoding the vaccine antigen. Specific examples of a vaccine are RNA-vaccines encoding the vaccine antigen. In particular, an RNA molecule can be used as a vaccine agent, in a naked form or formulated with a delivery vehicle. Specific embodiments may include a viral or bacterial host as gene delivery vehicle (e.g., live vaccine vector) or may include administering the gene in a free form, e.g., inserted into a plasmid. Specifically, the nucleic acid molecule encoding the vaccine antigen described herein is capable of expressing the fusion protein in a mammalian or human cell, and in particular upon vaccinating a subject.

The vaccine described herein preferably comprises an adjuvant. The vaccine antigen described herein may be formulated with specific adjuvants commonly used in human vaccines.

A specifically preferred adjuvant is selected from the group consisting of alum (aluminum phosphate gel or aluminum hydroxide gel or mixture of the two), AS04 (alum plus monophosphoryl lipid A), MF59 (oil-in-water emulsion adjuvant), and toll-like receptor agonist adjuvants (monophosphoryl lipid A plus CpG).

A suitable selection of adjuvants may include MF59, aluminum hydroxide, aluminum phosphate, calcium phosphate, cytokines (e.g. IL-2, IL-12, GM-CSF), saponins (e.g. QS21), MDP derivatives, CpG oligonucleotides, LPS, MPL, polyphosphazenes, emulsions (e.g. Freund's, SAF), liposomes, virosomes, ISCOMs, cochleates, PLG microparticles, poloxamer particles, virus-like particles, heat-labile enterotoxin (LT), cholera toxin (CT), mutant toxins (e.g., LTK63 and LTR72), microparticles and/or polymerized liposomes. Suitable adjuvants are commercially available as, for example, AS01B (MPL and QS21in a liposome formulation), AS02A, AS15, AS-2, AS-03 and derivatives thereof (GlaxoSmithKline, USA); CWS (cell-wall skeleton), TDM (trehalose-6,6′-dimycolate), LeIF (Leishmania elongation initiation factor), aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2,-7 or-12 may also be used as adjuvants. Preferred adjuvants for use in eliciting a predominantly Th1-type response include, for example, a combination of monophosphoryl lipid A, preferably 3-O-deacylated monophosphoryl lipid A (3D-MPL), optionally with an aluminum salt.

Another preferred adjuvant is a saponin or saponin mimetics or derivatives, preferably QS21 (Aquila Biopharmaceuticals Inc.), which may be used alone or in combination with other adjuvants. For example, an enhanced system involves the combination of a monophosphoryl lipid A and saponin derivative, such as the combination of QS21 and 3D-MPL. Other preferred formulations comprise an oil-in-water emulsion and tocopherol. A particularly potent adjuvant formulation is QS21, 3D-MPL and tocopherol in an oil-in-water emulsion. Additional saponin adjuvants for use in the present invention include QS7 (described in WO 96/33739 and WO 96/11711) and QS17 (described in U.S. Pat. No. 5,057,540 and EP 0 362 279 B1).

According to a specific aspect, the fusion protein is formulated with an adjuvant, preferably selected from the group consisting of alum, preferably aluminum phosphate gel or aluminum hydroxide gel or mixture of the two, AS04 (i.e., alum plus monophosphoryl lipid A), MF59 (i.e., an oil-in-water emulsion adjuvant), and toll-like receptor agonist adjuvants, such as monophosphoryl lipid A and CpG.

According to a specific aspect, the subject is on standard antiviral treatment, preferably with nucleos(t)ide (NUC) treatment.

According to a specific aspect, the subject has discontinued standard antiviral treatment, such as nucleos(t)ide (NUC) treatment e.g., 2 weeks-24 weeks prior to receiving the vaccine described herein.

According to a specific aspect, said treatment induces an anti-HBV and/or anti-HDV immune response, in particular an immune response that neutralizes HBV and/or HDV.

According to a specific aspect, said treatment induces a humoral anti-HBV and/or anti-HDV immune response. Specifically, upon treatment, the subject comprises a humoral anti-HBV immune response, in particular an anti-preS immune response, preferably an IgG immune response. Specifically, the level of serum anti-HBV antibodies is higher than before vaccination. An effective anti-HBV antibody level of e.g., at least any one of 2.5, 5 or 10 mIU/mL can be achieved, such as determined by a standard ELISA assay e.g., Abbott EIA AxSYM (Abbott, Abbott Park, IL, USA) can be used for the detection of HBsAg, or anti-HBV antibodies. According to protocols provided by the manufacturer, positive and negative cutoffs can be calculated with the positive and negative controls as required by the diagnostic kits.

Specifically, the immune response is neutralizing HBV. The HBV neutralizing antibodies are specifically immunoreactive with HBV serotypes and/or any one of the HBV genotypes B, C, D, E, F, G or H, or a subtype thereof.

According to a specific aspect, the subject is a patient chronically infected with HBV.

Specifically, the subject is a patient chronically infected with HBV and, upon treatment, mounts an immune response that neutralizes HBV.

According to a specific aspect, the invention further provides for a Hepatitis B virus (HBV) vaccine, for use in breaking immune tolerance in a human subject, by administering a vaccine comprising a preS polypeptide that is fused to at least one grass pollen allergen peptide, to induce HBV neutralizing antibodies. Specifically, the medical use is characterized as further described herein.

Specifically, the HBV vaccine is provided for use in inducing a preS-specific immune response in immunologically tolerant patients e.g., in patients with HBV and/or HDV infections, or subjects who are low or non-responders to S-protein-based HBV vaccines, by administering a vaccine comprising a preS polypeptide that is fused to at least one grass pollen allergen peptide, such as further described herein, to induce HBV neutralizing antibodies and/or HDV neutralizing antibodies, and/or to reduce the respective viral load e.g., the HBV viral load, as determined by a PCR assay.

### FIGURES

FIGS. 1-7 and 9-17: They contain VVX001 data (ClinicalTrials.gov Identifier: NCT03625934) as described in the Examples. VVX001 is identical to BM325 described in (Mechanisms, safety and efficacy of a B cell epitope-based vaccine for immunotherapy of grass pollen allergy. Zieglmayer P, Focke-Tejkl M, Schmutz R, Lemell P, Zieglmayer R, Weber M, Kiss R, Blatt K, Valent P, Stolz F, Huber H, Neubauer A, Knoll A, Horak F, Henning R, Valenta R. EBioMedicine. 2016 September; 11:43-57. doi:10.1016/j.ebiom.2016.08.022. Epub 2016 Aug. 20).

FIG. 1: preS-specific IgG levels (y-axis: OD values corresponding to specific IgG levels; x-axis: time points of serum sampling, V2 baseline sample) in VVX001-vaccinated (light grey) and placebo (dark grey) patients of patients from cohort 3 (i.e., patients who are chronically infected with HBV, but are classified as inactive carriers).

FIG. 2: VVX001 induced preS-specific IgG antibodies in naive subjects (cohort 1) and in chronically HBV-infected patients (cohort 3) in a comparable manner. PreS-specific antibody levels for the groups are shown on the y-axis at the different time points. Arrows indicate vaccinations.

FIG. 3: VVX001 induced a modest preS-specific CD4+ and CD8+ T cell response in naïve subjects (cohort 1). Shown are the percentages of proliferated CD4+ (left) and CD8+ T cells (right).

FIG. 4: Vaccination with VVX001 induces a modest preS-specific CD4+ responses and CD8+ T cell responses in chronic HBV patients (cohort 3). Shown are the percentages of proliferated CD4+ (left and CD8+ T cells (right).

FIG. 5: Vaccination with VVX001 induces virus-neutralizing antibodies in naive subjects (cohort 1) who have developed preS-specific antibodies. Shown are the levels of preS-specific IgG antibodies (OD levels) and the percentages of reduction of HBeAg and positive cells reflecting virus neutralization at the different time points (visits 2-8).

FIG. 6: Chronically infected HBV patients (cohort 3) who had low or no virus-neutralizing antibodies at visit 2 and developed preS-specific antibodies after vaccination with VVX001 develop or increase virus neutralizing antibodies. Shown are the levels of preS-specific IgG antibodies (OD levels) and the percentages of reduction of HBeAg and positive cells reflecting virus neutralization at the different time points (visits 2-8).

FIG. 7: Vaccination of a non-responder to protein S-based HBV vaccines with VVX001 induces CD4+ and CD8+ T cell responses to preS (black bars) and to a mix of preS-derived peptides (grey bars). Shown are the percentages of proliferated T cells (y-axes) after vaccination (black arrows) at different time points (x-axes).

FIG. 8: Sequences referred to herein.

FIG. 9: Vaccination of a non-responder to S-protein-based HBV vaccines with VVX001=BM325 (indicated) induces preS-specific IgG (y-axis: OD levels).

FIG. 10: Vaccination of a non-responder to S-protein-based HBV vaccines with VVX001=BM325 induces mainly preS-specific IgG and IgG1 (y-axis: OD levels).

FIG. 11: Overview of preS consisting of preS1 and preS2. The NTCP binding site is indicated in dark and light grey which is included in the genotype-specific peptides in FIG. 12.

FIG. 12: Vaccination of a non-responder to S-protein-based HBV vaccines with VVX001=BM325 induces IgG antibodies (y-axis: ODs corresponding to IgG levels) against peptides representing the different HBV genotypes containing the binding site for the sodium taurocholate co-transporting polypeptide (NTCP) receptor for HBV. The x-axis shows the time points (November 2018 before vaccination and then thereafter).

FIG. 13: Vaccination of non-responders (cohort 2) to S-protein-based HBV vaccines with VVX001=BM325 (indicated) but not with placebo induces preS-specific IgG antibodies (y-axis: OD levels). Time points are indicated on the x-axis.

FIG. 14: Vaccination of non-responders (cohort 2) to S-protein-based HBV vaccines with VVX001=BM325 (indicated) but not with placebo induces preS-specific virus-neutralizing IgG antibodies (y-axis: percentage of HBeAg reduction corresponding to virus neutralization). Time points are indicated on the x-axis.

FIG. 15: Vaccination of chronically HBV infected patients under NUC therapy with VVX001=BM325 (indicated) but not with placebo induces preS-specific IgG antibodies (y-axis: OD values corresponding to IgG levels). Time points are indicated on the x-axis. NUCs were removed at visit 5.

FIG. 16: Vaccination of chronically HBV infected patients under NUC therapy with VVX001=BM325 (indicated) but not with placebo induces preS-specific virus-neutralizing IgG antibodies (y-axis: HBeAg reduction corresponding to virus neutralization). Time points are indicated on the x-axis. NUCs were removed at visit 5.

FIG. 17: Vaccination of chronically HBV infected patients under NUC therapy with VVX001=BM325 (indicated) but not with placebo induces preS-specific CD4+ (left) and CD8+ (right) T cell responses (y-axis: percentages proliferated cells). Time points are indicated on the x-axis. NUCs were removed at visit 5.

### DETAILED DESCRIPTION OF THE INVENTION

Specific terms as used throughout the specification have the following meaning.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−10% or +/−5% of the given value.

The term “antigen” (herein also referred to as “immunogen”) as used herein, refers to any molecule that is recognized by the immune system and that can stimulate an immune response. In some embodiments, the antigen is a polypeptide or protein, and in particular a component of an infectious agent.

The term “antigen” as used herein shall in particular refer to any antigenic determinant, which can be possibly recognized by a binding site of an antibody or is able to bind to the peptide groove of HLA class I or class Il molecules and as such may serve as stimulant for specific T cells. The antigen is either recognized as a whole molecule or as a fragment of such molecule, especially substructures e.g., a polypeptide or carbohydrate structure, generally referred to as “epitopes” e.g., B cell epitopes, T cell epitope), which are immunologically relevant i.e., are also recognizable by natural or monoclonal antibodies.

Specifically, preferred antigens are those molecules or structures, which have already been proven to be or are capable of being immunologically or therapeutically relevant, especially those, for which a clinical efficacy has been tested. The term as used herein shall in particular comprise molecules or structures selected from antigens comprising immuno-accessible and immuno-relevant epitopes, in particular conserved antigens found in one or more species or serotype. Immuno-accessible viral epitopes are typically presented by or comprised in antigens expressed on the outer surface of a virion or on the surface of an infected cell.

Selected epitopes and polypeptides as described herein may trigger an immune response in vivo, so to induce neutralizing antibodies against the antigen and target virus, respectively. This provides for the effective protection upon active immunization with the antigen. Polypeptide antigens are preferred antigens due to their inherent ability to elicit both cellular and humoral immune responses.

The term “epitope” as used herein shall in particular refer to a molecular structure which may completely make up a specific binding partner or be part of a specific binding partner to a binding site of an antibody. Chemically, an epitope recognized by antibodies may either be composed of a peptide, a carbohydrate, a fatty acid, an organic, biochemical or inorganic substance or derivatives thereof and any combinations thereof. If an epitope is a polypeptide, it will usually include at least 3 amino acids, preferably at least 4, 5, 6, 7, 8, 9, 10, 11, 12 or 13 amino acids. There is no critical upper limit to the length of the peptide, which could comprise nearly the full length of a polypeptide sequence of a protein. Epitopes can be either linear, sequential or discontinuous and if they assemble a structure can be conformational epitopes. A linear epitope is comprised of a single segment of a primary sequence of a polypeptide or carbohydrate chain. Discontinuous conformational epitopes are comprised of amino acids or carbohydrates brought together by folding of the polypeptide to form a tertiary structure and the amino acids are not necessarily adjacent to one another in the linear sequence.

The terms “immune tolerance”, “immunological tolerance”, or “immunotolerance” are herein synonymously used and shall refer to a state of unresponsiveness of the immune system so that no adaptive (i.e., antibody, T cell responses) are mounted to certain antigens, substances or tissues.

The term “isolated” or “isolation” as used herein with respect to a polypeptide, protein or nucleic acid molecule such as the vaccine antigen and the nucleic acid molecule(s) encoding such vaccine antigen as described herein, shall refer to such compound that has been sufficiently separated from the environment with which it would naturally be associated, so as to exist in “purified” or “substantially pure” form. Yet, “isolated” does not necessarily mean the exclusion of artificial or synthetic fusions or mixtures with other compounds or materials, or the exclusion of impurities that do not interfere with the fundamental activity, and that may be present, for example, due to incomplete purification. Isolated compounds can be further formulated to produce preparations thereof, and still for practical purposes be isolated-for example, a set of peptides or the respective peptide fusions described herein can be mixed with pharmaceutically acceptable carriers, including those which are suitable for analytic, diagnostic, prophylactic or therapeutic applications, or excipients when used in diagnosis, medical treatment, or for analytical purposes.

The term “purified” as used herein shall refer to a preparation comprising at least 50% (w/w total protein), preferably at least 60%, 70%, 80%, 90% or 95% of a compound (e.g., a vaccine antigen described herein). A highly purified product is essentially free from contaminating proteins, and preferably has a purity of at least 70%, more preferred at least 80%, or at least 90%, or even at least 95%, up to 100%. Purity is measured by methods appropriate for the compound (e.g., chromatographic methods, polyacrylamide gel electrophoresis, HPLC analysis, and the like). An isolated, purified vaccine antigen described herein may be obtained as a recombinant product obtained by purifying from a host cell culture expressing the product in the cell culture supernatants, to reduce or remove host cell impurities or from cellular debris.

As isolation and purification methods for obtaining a purified polypeptide or protein product, methods utilizing difference in solubility, such as salting out and solvent precipitation, methods utilizing difference in molecular weight, such as ultrafiltration and gel electrophoresis, methods utilizing difference in electric charge, such as ion-exchange chromatography, methods utilizing specific affinity, such as affinity chromatography, methods utilizing difference in hydrophobicity, such as reverse phase high performance liquid chromatography, and methods utilizing difference in isoelectric point, such as isoelectric focusing may be used. The following standard methods can be used: cell (debris) separation and wash by Microfiltration or Tangential Flow Filter (TFF) or centrifugation, protein purification by precipitation or heat treatment, protein activation by enzymatic digest, protein purification by chromatography, such as ion exchange (IEX), hydrophobic interaction chromatography (HIC), Affinity chromatography, size exclusion (SEC) or HPLC chromatography, protein precipitation of concentration and washing by ultrafiltration steps. An isolated and purified protein can be identified by conventional methods such as Western blot, HPLC, activity assay, or ELISA.

With reference to nucleic acid molecules as described herein, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA (e.g., mRNA) molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

With reference to polypeptides or proteins, the term “isolated” shall specifically refer to compounds that are free or substantially free of material with which they are naturally associated such as other compounds with which they are found in their natural environment, or the environment in which they are prepared (e g. cell culture) when such preparation is by recombinant DNA technology practiced in vitro or in vivo. Isolated compounds can be formulated with diluents or adjuvants and still for practical purposes be isolated-for example, the polypeptides or polynucleotides can be mixed with pharmaceutically acceptable carriers or excipients when used in diagnosis or therapy.

The term “nucleic acid molecule” used herein refers to either DNA (including e.g., cDNA) or RNA (including e.g., mRNA) molecules comprising a polynucleotide sequence. The molecule may be a single or double-stranded polymer of deoxyribonucleotide or ribonucleotide bases read from the 5′ to the 3′ end. The term includes coding sequences, such as genes, artificial polynucleotides such as comprised in an expression construct expressing the respective polypeptide sequence. A DNA or RNA molecule can be used which comprises a nucleotide sequence that is degenerate to any of the sequences or a combination of degenerate sequences, or which comprises a codon-optimized sequence to improve expression in a host. For example, a specific eukaryotic host cell codon-optimized sequence can be used. Specific RNA molecules can be used to provide a respective RNA-vaccine.

A recombinant nucleic acid may be one that has a sequence that is not naturally occurring or that has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques well-known in the art. For example, a nucleic acid can be chemically synthesized using naturally-occurring nucleotides or variously modified nucleotides designed to increase the biological stability of the molecules or to increase the physical stability of the duplex formed upon hybridization.

Mutants of a naturally-occurring or natural protein or polypeptide as naturally-occurring in a wild-type source virus such as HBV, like a HBV preS polypeptide, or mutants of grass pollen allergen peptides as described herein may be provided e.g., by introducing a certain number of point mutations into a parent amino acid sequence. Specifically, a mutagenesis method is used to introduce one or more point mutations.

A point mutation as described herein is typically at least one of a deletion, insertion, and/or substitution of one or more nucleotides within a nucleotide sequence to achieve the deletion, insertion, and/or substitution of one (only a single one) amino acid at a certain, defined position within the amino acid sequence encoded by said nucleotide sequence. Therefore, the term “point mutation” as used herein shall refer to a mutation of a nucleotide sequence or an amino acid sequence. Specifically, preferred point mutations are substitutions, in particular conservative ones. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc. Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

Specific mutagenesis methods provide for point mutations of one or more nucleotides in a sequence, in some embodiments tandem point mutations, such as to change at least or up to 2, 3, 4, or 5 contiguous nucleotides within a nucleotide sequence of a parent molecule.

The term “mutagenesis” as used herein shall refer to a method of preparing or providing mutants of a nucleotide sequence and the respective protein encoded by said nucleotide sequence e.g., through insertion, deletion and/or substitution of one or more nucleotides, so to obtain variants thereof with at least one change in the coding region. Mutagenesis may be through random, semi-random or site directed mutation. A mutagenesis method can encompass methods of engineering the nucleic acid or de novo synthesizing a nucleotide sequence using the respective parent sequence information as a template. By a method of mutagenesis, synthetic polynucleotides or genes can be produced which code for a desired polypeptide or recombinant fusion protein.

Any of the exemplary proteins or polypeptides described herein may e.g., be used as a parent molecule and be modified to produce variants and mutants, which have substantially the same or an even improved immunogenic effect as the parent one, or which may include one or more point mutations which are also found in one or more different wild-type mutants of a virus. For instance, a library of nucleotide sequences may be prepared by mutagenesis of a selected parent nucleotide sequence encoding a protein or polypeptide originating from a wild-type source virus such as HBV, or originating from a wild-type grass pollen allergen. A library of variants may be produced and a suitable mutant of the respective protein or polypeptide be selected according to a specifically desired genotype or phenotype.

As used herein, the term “mutant” with respect to a virus species or a viral protein, also referred to as “variant”, shall include all naturally-occurring or artificial compounds which differ from the respective original (parent) compound by at least one mutation that changes the structure or amino acid sequence of the parent compound. Mutants may differ in at least one amino acid that may change immunogenicity or the respective antibody response, such that antibodies induced by the parent compound no more recognize a mutant compound. To cover such mutants, it is preferred to mutagenize a parent vaccine antigen (or a part thereof e.g., said preS polypeptide comprised in the vaccine antigen) such that all relevant point mutations that naturally-occur in one or more (a variety) of mutants be comprised in the mutagenized vaccine antigen, with the effect of inducing an immune response that covers not only the source virus of the parent vaccine antigen (or the part thereof originating from such source virus), but also the respective mutant virus(es) which are characterized by one or more of said relevant point mutations.

The term “naturally-occurring” as used herein with respect to a protein or polypeptide, or a specific point mutation, is understood to be found (occur) in a wild-type organism or virus (including wild-type mutant viruses). Mutants may be naturally-occurring or artificial. Naturally-occurring (also referred to as “wild-type”) proteins or polypeptides are herein also referred to as being “natural”. The present disclosure specifically refers to natural preS polypeptide and grass pollen allergen peptides, including e.g., naturally-occurring mutants thereof. A point mutation is understood as a naturally-occurring point mutation if also comprised in a natural protein or polypeptide originating from a mutant natural source.

The term “neutralizing” as used herein with respect to antibodies against a target virus is herein understood as follows. Specifically, neutralizing antibodies prevent HBV from infecting the corresponding host cell. HBV neutralizing antibodies as described herein, due to their specific function, are expected to protect the host from getting infected with the virus, or to protect chronically diseased patients from reinfection of their host cells to reduce the viral load and/or to protect against recurrent HBV disease. Reduction of HBV viral load can be determined by the reduction of HBSAg or viral DNA in positive subjects.

Neutralizing HBV antibodies can be tested by standard virus neutralization tests (VNTs). Neutralizing activity against a virus strain can be tested in cell-based assays and in vivo. Neutralizing antibodies can be determined e.g., by enumerating virus titers in the presence of antibodies and detecting cytopathic effect in cell-based infection assays. (Immunotherapy With the preS-based Grass Pollen Allergy Vaccine BM32 Induces Antibody Responses Protecting Against Hepatitis B Infection. Cornelius C, Schöneweis K, Georgi F, Weber M, Niederberger V, Zieglmayer P, Niespodziana K, Trauner M, Hofer H, Urban S, Valenta R. EBioMedicine. 2016 September; 11:58-67. doi:10.1016/j.ebiom.2016.07.023. Epub 2016 Aug. 8;).

According to a specific embodiment, the vaccine antigen described herein is produced as a recombinant polypeptide, such as produced by recombinant DNA technology.

As used herein, the term “recombinant” refers to a molecule or construct that does not naturally occur in a host cell. In some embodiments, recombinant nucleic acid molecules contain two or more naturally-occurring sequences that are linked together in a way that does not occur naturally. A recombinant protein refers to a protein that is encoded and/or expressed by a recombinant nucleic acid. In some embodiments, “recombinant cells” express genes that are not found in identical form within the natural (i.e., non-recombinant) form of the cell and/or express natural genes that are otherwise abnormally over-expressed, under-expressed, and/or not expressed at all due to deliberate human intervention. Recombinant cells contain at least one recombinant polynucleotide or polypeptide. “Recombination”, “recombining”, and generating a “recombined” nucleic acid generally encompass the assembly of at least two nucleic acid fragments.

The term “recombinant” as used herein specifically means “being prepared by or the result of genetic engineering” i.e., by human intervention. A recombinant nucleotide sequence may be engineered by introducing one or more point mutations in a parent nucleotide sequence, and may be expressed in a recombinant host cell that comprises an expression cassette including such recombinant nucleotide sequence. The polypeptide expressed by such expression cassette and host cell, respectively, is also referred to as being “recombinant”. For the purpose described herein conventional molecular biology, microbiology, and recombinant DNA techniques within the skill of the art may be employed. Specific embodiments described herein refer to the production of a vaccine antigen, and the recombinant means for such production, including a nucleic acid encoding the amino acid sequence, an expression cassette, a vector or plasmid comprising the nucleic acid encoding the amino acid sequence to be expressed, and a host cell comprising any such means. Suitable standard recombinant DNA techniques are known in the art and described inter alia in Sambrook et al., “Molecular Cloning: A Laboratory Manual” (1989), 2nd Edition (Cold Spring Harbor Laboratory press).

Methods for the production of fusion proteins are well known in the art and can be found in standard molecular biology references such as Sambrook et al. (Molecular Cloning, 2^{nd} ed., Cold Spring Harbor Laboratory Press, 1989) and Ausubel et al. (Short Protocols in Molecular Biology, 3^{rd} ed; Wiley and Sons, 1995). Typically, a fusion protein is produced by first constructing a fusion gene which is inserted into a suitable expression vector, which is, in turn, used to transfect a suitable host cell. Recombinant fusion constructs can be produced by a series of restriction enzyme digestions and ligation reactions which result in the desired sequences being incorporated into a plasmid, or by specific gene editing techniques. Synthetic oligonucleotide adapters or linkers can be used as is known by those skilled in the art and described in the references cited above. The elements of a fusion protein to be fused can be assembled prior to insertion into a suitable expression construct or vector. Insertion of the sequence within the vector should be in frame so that the sequence can be transcribed into a protein. The assembly of DNA constructs is routine in the art and can be readily accomplished by a person skilled in the art.

The term “sequence identity” of a variant or mutant as compared to a parent nucleotide or amino acid sequence indicates the degree of identity of two or more sequences. Two or more amino acid sequences may have the same residues at a corresponding position, to a certain degree, up to 100%. Two or more nucleotide sequences may have the same or conserved base pairs at a corresponding position, to a certain degree, up to 100%.

Sequence similarity searching is an effective and reliable strategy for identifying homologs with excess (e.g., at least 80%) sequence identity. Sequence similarity search tools frequently used are e.g., BLAST, FASTA, and HMMER.

The term “percent (%) amino acid sequence identity” as used herein with respect to an amino acid sequence, is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

For purposes described herein, the sequence identity between two amino acid sequences can be determined using the NCBI BLAST program version BLASTP 2.8.1 with the following exemplary parameters: Program: blastp, Word size: 6, Expect value: 10, Hitlist size: 100, Gapcosts: 11.1, Matrix: BLOSUM62, Filter string: F, Compositional adjustment: Conditional compositional score matrix adjustment.

For pairwise protein sequence alignment of two amino acid sequences along their entire length the EMBOSS Needle webserver (Pairwise Sequence Alignment; EMBL-EBI, Wellcome Genome Campus, Hinxton, Cambridgeshire, CB10 1SD UK,) was used with default settings (Matrix: EBLOSUM62; Gap open: 10; Gap extend: 0.5; End Gap Penalty: false; End Gap Open: 10; End Gap Extend: 0.5). EMBOSS Needle uses the Needleman-Wunsch alignment algorithm to find the optimum alignment (including gaps) of the two input sequences and writes their optimal global sequence alignment to file.

The term “percent (%) identity” as used herein with respect to a nucleotide sequence, is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

For purposes described herein (unless indicated otherwise), the sequence identity between two amino acid sequences can be determined using the NCBI BLAST program version BLASTN 2.8.1 with the following exemplary parameters: Program: blastn, Word size: 11, Expect threshold: 10, Hitlist size: 100, Gap Costs: 5.2, Match/Mismatch Scores: 2,-3, Filter string: Low complexity regions, Mark for lookup table only.

Herein the term “subject” is herein understood to refer to human subjects, in particular human beings who are either patients suffering from a specific disease condition or healthy subjects. In particular the treatment and medical use described herein applies to a subject in need of prophylaxis or therapy of a disease condition associated with a HBV infection or a HBV disease. Specifically, the treatment may be by inducing an immune response interfering with the pathogenesis of a disease condition where HBV is a causal agent of the condition. The subject may be a subject at risk of such disease condition or suffering from disease.

The term “at risk of” a certain disease conditions, refers to a subject that potentially develops such a disease condition e.g., by a certain predisposition, exposure to virus or virus-infected subjects, or that already suffers from such a disease condition at various stages, particularly associated with other causative disease conditions or else conditions or complications following as a consequence of viral infection.

The term “patient” includes human subjects that receive either prophylactic or therapeutic treatment. Subjects described herein may be patients or healthy subjects.

The term “treatment” as used herein shall always refer to treating subjects for prophylactic (i.e., to prevent infection and/or disease status) or therapeutic (i.e., to treat diseases regardless of their pathogenesis) purposes. The vaccine antigen described herein is specifically provided for active immunotherapy.

Specifically, the term “prophylaxis” refers to preventive measures which is intended to encompass prevention of the onset of pathogenesis or prophylactic measures to reduce the risk of pathogenesis.

The term “therapy” as used herein with respect to treating subjects refers to medical management of a subject with the intent to cure, ameliorate, stabilize, reduce the incidence or prevent a disease, pathological condition, or disorder, which individually or together are understood as “disease condition”.

The vaccine described herein specifically comprises the vaccine antigen in an effective amount, which is herein specifically understood as “immunologically effective amount”.

By “immunologically effective amount”, it is meant that the administration of that amount to a subject, either in a single dose or as part of a series of doses, is effective on the basis of the therapeutic or prophylactic treatment objectives. A “prophylactically effective amount” refers to an amount effective, at dosages and for periods of time necessary, to achieve the desired prophylactic result, such as preventing a target virus infection, or inhibiting a target virus disease onset or progression. This amount will vary depending upon the health and physical condition of the subject to be treated, age, the capacity of the subject's immune system to synthesize antibodies, the type and degree of immune response desired, the formulation of the vaccine, and other conditions.

An effective amount or dosage is typically capable of eliciting an immune response in a subject of effective levels of antibody titer to bind and neutralize a target virus species e.g., after a first or repeated immunization. The effectiveness can be assayed by the respective antibody titers in samples of blood taken from the subject and in particular by measuring neutralizing antibodies. It may be also done by measuring virus-specific T cell responses.

In some embodiments, an effective amount is one that has been correlated with beneficial effect when administered as part of a particular dosing regimen e.g., a single administration or a series of administrations such as in a “boosting” regimen. For treatment, the vaccine described herein may be administered at once, or may be divided into the individual components and/or a number of smaller doses to be administered at intervals of time. Typically, upon priming a subject by a first injection of a vaccine according to the invention, one or more booster injections may be performed over a period of time by the same or different administration routes. Where multiple injections are used, subsequent injections may be made e.g., within 1 to 52 weeks of the previous injection.

The vaccine described herein may comprise the vaccine antigen in an immunogenic formulation. Specific embodiments comprise one or more adjuvants and/or pharmaceutically acceptable excipients or carriers.

Pharmaceutical carriers suitable for facilitating certain means of administration are well known in the art. Specific embodiments refer to immunogenic formulations, which comprise a pharmaceutically acceptable carrier and/or adjuvant, which trigger a humoral (B cell, antibody), helper or cytotoxic (T cell) immune response. Adjuvants may specifically be used to enhance the effectiveness of the vaccine. Adjuvants may be added directly to the vaccine compositions or can be administered separately, either concurrently with or shortly before or after administration of the vaccine antigen.

The term “adjuvant” as used herein specifically refers to a compound that when administered in conjunction with an antigen, augments and/or redirects the immune response to the antigen, but when administered alone does not generate an immune response to the antigen. Adjuvants can augment an immune response by several mechanisms including lymphocyte recruitment, stimulation of B cells and/or T cells, and stimulation of macrophages and other antigen presenting cells, such as, e.g., dendritic cells.

An “effective amount” of an adjuvant can be used in a vaccine described herein, which is specifically understood to be an amount which enhances an immunological response to the immunogen such that, for example, lower or fewer doses of the immunogenic composition are required to generate a specific immune response and a respective effect of preventing or combating virus infection or disease.

Pharmaceutically acceptable carriers generally include any and all suitable solvents, dispersion media, coatings, antibacterial and antifungal agents, isotonic and absorption delaying agents, and the like that are physiologically compatible with an antibody or related composition or combination provided by the invention. Specific examples of pharmaceutically acceptable carriers include sterile water, saline, phosphate buffered saline, dextrose, glycerol, ethanol, polyethylene glycol, and the like, as well as combinations of any thereof. Additional pharmaceutically acceptable carriers are known in the art and described in, e.g., Remington: The Science and Practice of Pharmacy, 22^{nd} revised edition (Allen Jr, LV, ed., Pharmaceutical Press, 2012). Liquid formulations can be solutions, emulsions or suspensions and can include excipients such as suspending agents, solubilizers, surfactants, preservatives, and chelating agents. Exemplary carriers are liposomes or cationic peptides.

The preferred preparation is in a ready-to-use, storage stable form, with a shelf-life of at least one or two years. The invention also provides a delivery device e.g., a syringe, pre-filled with the vaccine described herein.

The vaccine described herein can be administered by conventional routes known within/to the vaccine field, such as via a parenteral (e.g., subcutaneous, intradermal, intramuscular, intravenous, or intraperitoneal) route, to a mucosal (e.g., ocular, intranasal, pulmonary, oral, gastric, intestinal, rectal, vaginal, or urinary tract) surface, or by topical administration to the skin (e.g., via a patch). The choice of administration route depends upon a number of parameters, such as the adjuvant used in the vaccine. If a mucosal adjuvant is used, the intranasal or oral route is preferred. If a lipid formulation or an aluminum compound is used, the parenteral route is preferred with the sub-cutaneous or intramuscular route being most preferred. The choice also depends upon the nature of the vaccine agent.

The foregoing description will be more fully understood with reference to the following examples. Such examples are, however, merely representative of methods of practicing one or more embodiments of the present invention and should not be read as limiting the scope of invention.

### EXAMPLES

### Example 1: Data are in Part from a Study to Evaluate Induction of HBV Virus Neutralizing Antibodies Using VVX001, ClinicalTrials.gov Identifier: NCT03625934

Brief Summary: The Study is evaluating the effects of VVX001, a vaccine against hepatitis B, to elicit a robust protective IgG immune response in vaccine naive subjects in subjects who failed to demonstrate seroconversion after treatment with a licensed hepatitis B vaccine and in patients chronically infected with HBV.

VVX001 is a fusion protein composed of preS from the large surface antigen of HBV and peptides derived from the grass pollen allergen Phl p 5 (Viravaxx AG), SEQ ID NO: 13. VVX001 was tested to show an immune reaction in patients who had failed to mount HBV-specific immune responses after HBV vaccination (cohort 2) or were chronically infected with the hepatitis B virus (HBV) (cohorts 3 and 4a) in a clinical trial.

The exemplary formulation comprises 0.05 mg of VVX001/mL adsorbed to 1.5 mg/ml aluminum hydroxide. The vaccine is filled in a syringe with aliquots of 550 μL. A dose of 20 μg is provided in 400 μL. The vaccine is repeatedly administered by subcutaneous injections, five injections at intervals of 4 weeks (+/−5 days).

The N-terminal part of the large surface antigen of the hepatitis B virus (preS), comprising amino acids 1-155 (serotype A3) is used. The selection process, which optimized the vaccine for use in AIT for grass pollen allergy has been described in detail in (Focke-Tejkl et al. J. Allergy Clin. Immunol. 2015; 135:1207-1217).

Mechanism of action of VVX001: The intended mechanism of action of allergy vaccines based on the Peptide/Carrier concept is the induction of protective blocking antibodies. In addition to IgG antibodies directed against grass pollen allergens, was observed in animal models that the vaccine and its components were also able to potently elicit an IgG antibody response to the carrier protein preS.

Using epitope mapping of preS, it could be demonstrated that this IgG response is focussed on the epitope of HBV responsible for binding to the hepatocyte NTCP protein, which is required for virus entry into the target cell. It was shown that these antibodies are functional for neutralizing the hepatitis B virus. In a cell culture model, they were able to block virus infection of susceptible hepatic cells.

Methods: VX001 has been investigated in 4 cohorts (Cohort 1: vaccination naïve healthy volunteers; cohort 2: HBV vaccination non responders, cohort 3: patients with chronic HBV infection; cohort 4: chronic hepatitis B patients on NUC's who discontinue their NUC therapy prior to vaccination with VVX001).

Cohort 1: Hepatits B vaccine naive subjects. Seronegative for anti-HBs and anti-HBc antibodies and for HBsAg.

Cohort 2: Healthy subjects failing to achieve seroprotection (non-responders). Subjects who failed to develop a protective immune response upon standard vaccination protocols with licensed hepatitis B vaccination (<10 IU/L anti-HBs antibodies). Seronegative for anti-HBs (<10 IU/L) and anti-HBc and for HBsAg.

Cohort 3: Patients chronically infected with HBV, without active hepatitis (inactive carriers). Parameters confirmed at screening during the past 12 months
HBeAg negative; HBsAg positive at screening <3000 IU/ml;
HBV viral load <2000 IU/ml
ALT Levels ≤ULN at screening

Cohort 4: Patients with chronic hepatitis B infection on current antiviral treatment (depending on time of cessation of antiviral treatment, the cohort 4 is divided in 4a and 4b).

Cohort 4a: Parameters confirmed at screening during the last 12 months
HBeAg negative; HBsAg positive <1000 IU/ml
HBV DNA not detectable for at least 2 years.
History of nucleos (t) die Treatment for at least 3 years
Willingness to discontinue NUC treatment during study
ALT levels ≤ULN at screening

Cohort 4b: in addition to cohort 4a:
willingness to discontinue NUC treatment 6 weeks before entering the Study.
ALT Levels ≤ULN 6 weeks before entering the study and
5× ULN at screening

Participants have been randomized (3:1) to receive 5 sc. injections of either (20 μg of VVX001 in 400 μL) VVX001 or placebo, (adjuvant Al(OH)₃), at each visit 4 weeks apart. After completion of the treatment phase patients were followed until week 52 (visit 7,8,9).

PreS was expressed in E. coli and preS-specific IgG response was measured by ELISA (Tulaeva et al, EBioMedicine 2020;59:102953). The assessment of CD4+/CD8+ lymphocyte proliferation to preS was performed with freshly isolated PBMCs at visits 2, 3, 5, 7, 8, 9 using carboxyfluorescein succinimidyl ester (CFSE) labelling (Cornelius et al. EBioMedicine 2016 September; 11:58-67).

Results: Vaccination with VVX001:
Safe for cohort 1, 2, 3 and 4a subjects
Induces a robust preS-specific IgG response in cohort 1, 2, 3 and 4a subjects.
Induces a modest CD4+ and CD8+ T cell response in in cohort 1, 2, 3 and 4a subjects
Induces virus-neutralizing antibodies in in cohort 1, 2, 3 and 4a subjects.

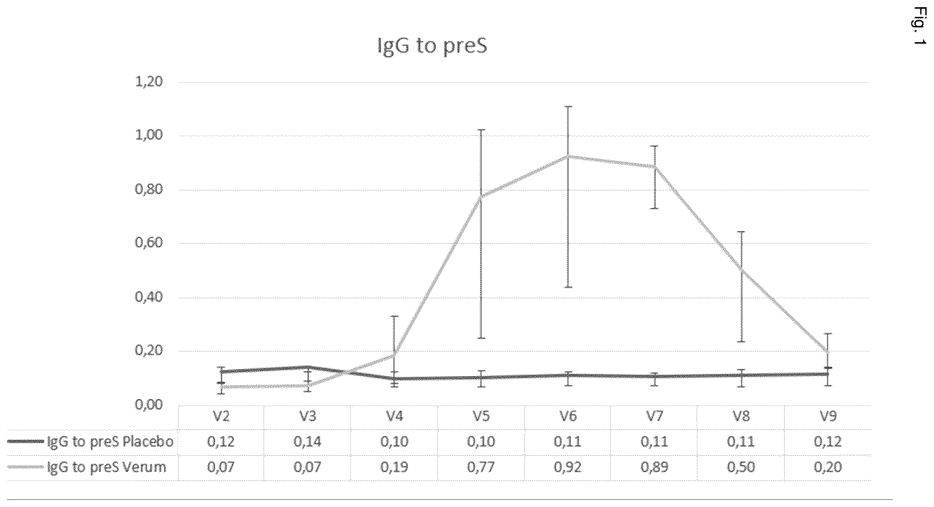

FIG. 1 shows that cohort 3 patients who are inactive carriers of HBV do not mount preS-specific IgG antibodies before vaccination with VVX001 and that vaccination with VVX001 induces the development of preS-specific IgG antibodies whereas no preS-specific IgG antibodies developed in cohort 3 patients vaccinated only with placebo. Thus, VVX001 can overcome immune tolerance to preS in chronically infected HBV patients who are carriers of the virus.

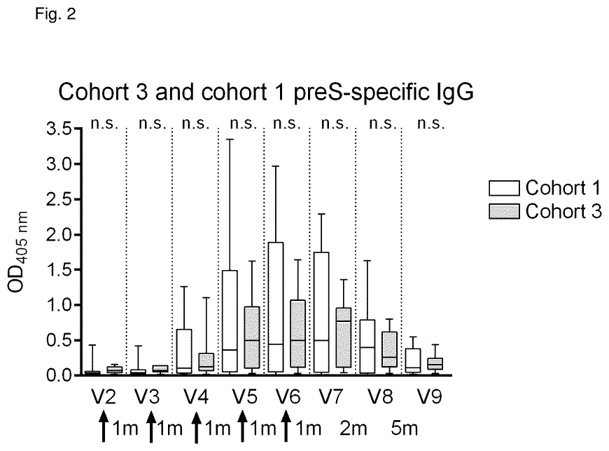

FIG. 2 illustrates that the preS-specific IgG response in VVX001-vaccinated cohort 3 subjects is of similar magnitude as in VVX001-vaccinated HBV-naïve subjects.

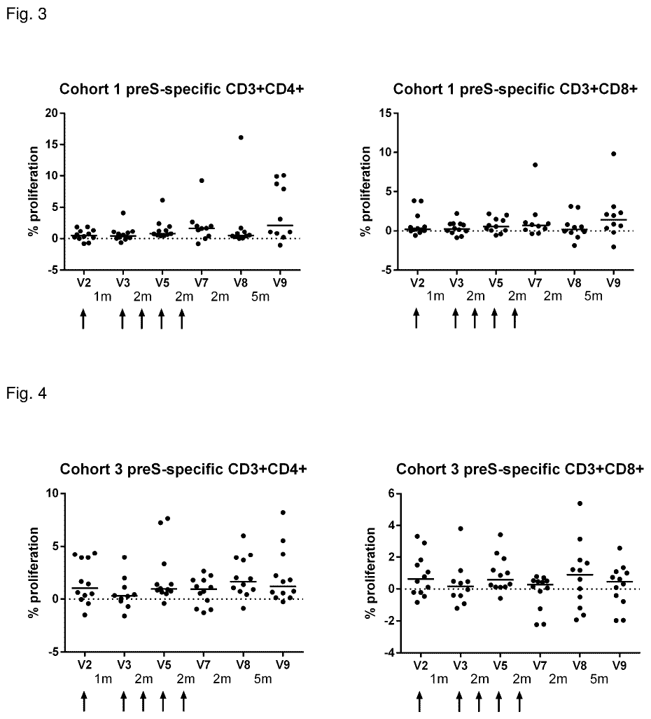

FIGS. 3 and 4 show that the induction of preS-specific IgG antibodies in VVX001-vaccinated subjects from cohort 1 and cohort 3 is accompanied by a modest increase of preS-specific CD4+ and CD8+ T cell responses, respectively.

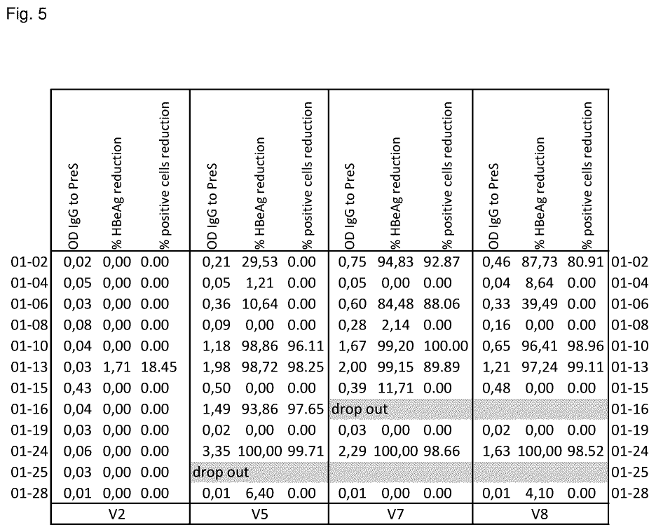

FIG. 5 demonstrates that the induction of preS-specific IgG responses in VVX001-vaccinated cohort 1 patients are accompanied by the development of virus neutralization capacity due to these antibodies.

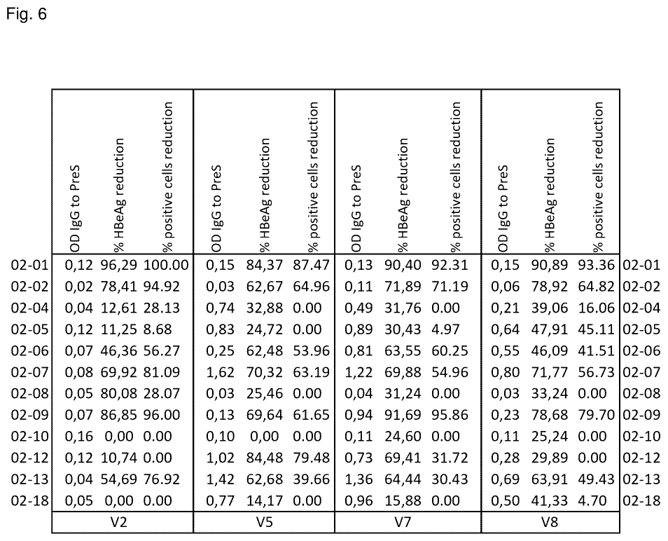

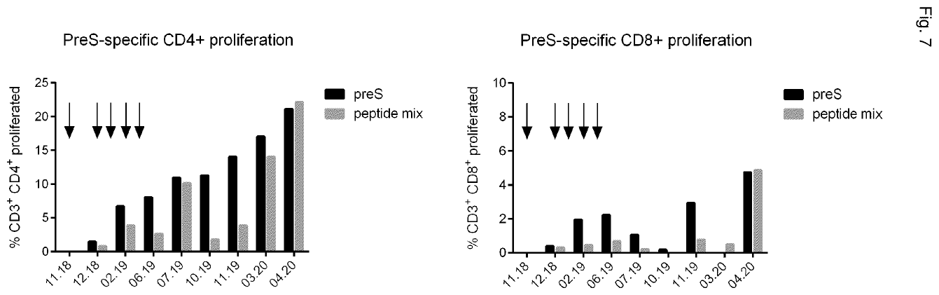

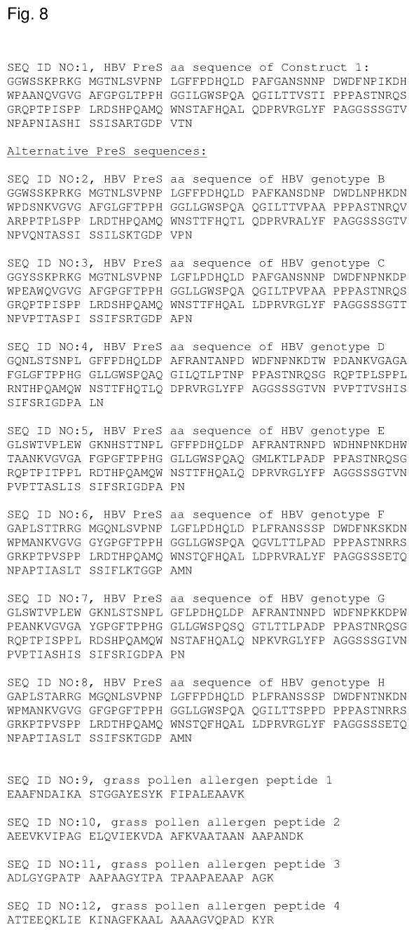

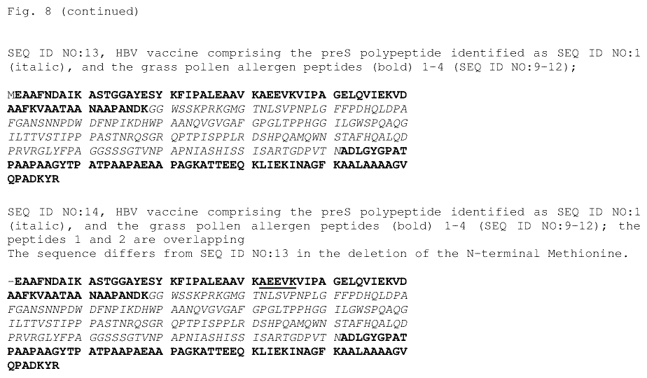

FIG. 6 shows that chronically infected HBV patients with an inactive virus carrier status not requiring NUC treatment partly contain virus-neutralizing antibodies already at baseline before vaccination. Since these subjects did not have preS-specific antibodies at the time point V2, the neutralizing antibodies present at this time point must be directed to the S antigen. Cohort 3 subjects who had low (e.g., 02-04, 02-05, 02-12) or no (e.g., 02-18) neutralizing antibodies, developed upon vaccination with VVX001 preS-specific IgG and thus mounted or increased their virus-neutralization capacity.

FIG. 7 demonstrates that vaccination of a non-responder to HBV vaccines induced the development and proliferation of preS-specific CD4+ and CD8+ T cell responses overcoming HBV-specific immune tolerance in this subject.

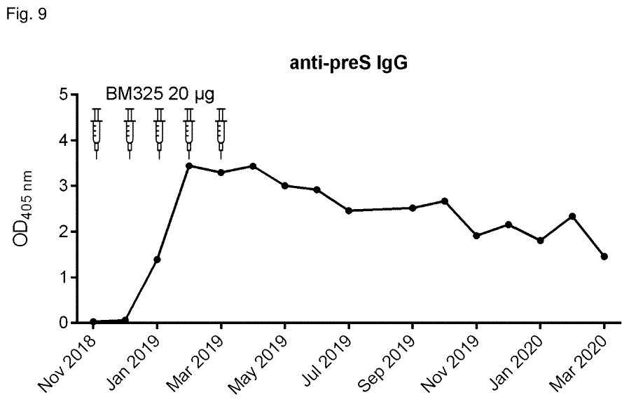

Accordingly, FIG. 9 shows that VVX001 could induce the development of preS-specific IgG in the aforementioned non-responder, as a demonstration that not only HBV-specific T cell tolerance but also B cell tolerance could be overcome.

Table 1 shows the development of preS-specific IgG₁ and IgG₄ antibodies in the VVX001-vaccinated subject from FIGS. 7 and 9.

TABLE 1
Quantification of preS-specific IgG1 and
IgG4 antibody responses [microgram/ml]

| | | Anti-preS | Anti-preS |
|---|---|---|---|
| Time point | Date | IgG₁, μg/ml | IgG₄, μg/ml |
| Baseline | 19 Nov. 2018 | 6.19 | 0 |
| 1 month post | 5 Apr. 2019 | 1940.72 | 15 |
| 5^{th} injection | | | |
| 4 months post | 26 Jun. 2019 | 1347.55 | 16.4 |
| 5^{th} injection | | | |
| 8 months post | 7 Nov. 2019 | 868.05 | 4.4 |
| 5^{th} injection | | | |
| 12 months post | 12 Mar. 2020 | 679.51 | 0 |
| 5^{th} injection | | | |

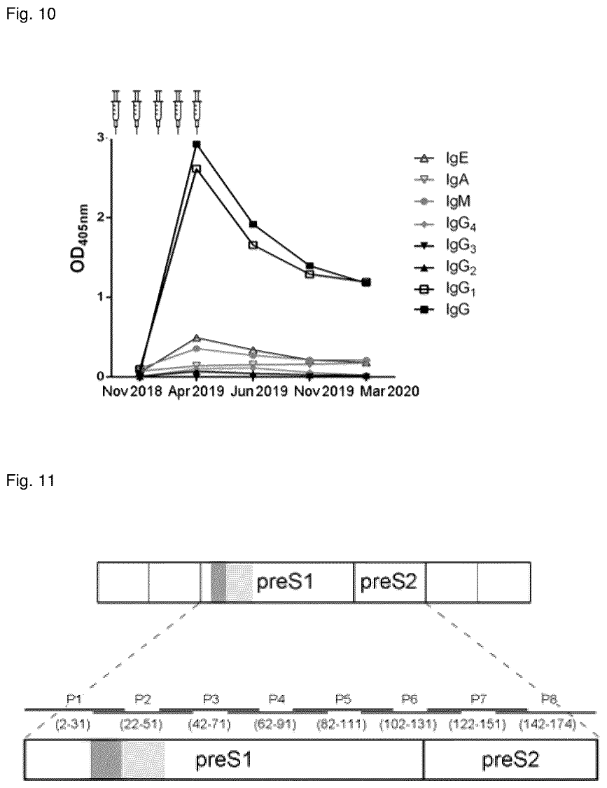

FIG. 10 and Table 1 illustrate that the majority of preS-specific IgG antibodies induced in the non-responder belonged to the IgG₁ subclass but there was also an induction of preS-specific IgG₄.

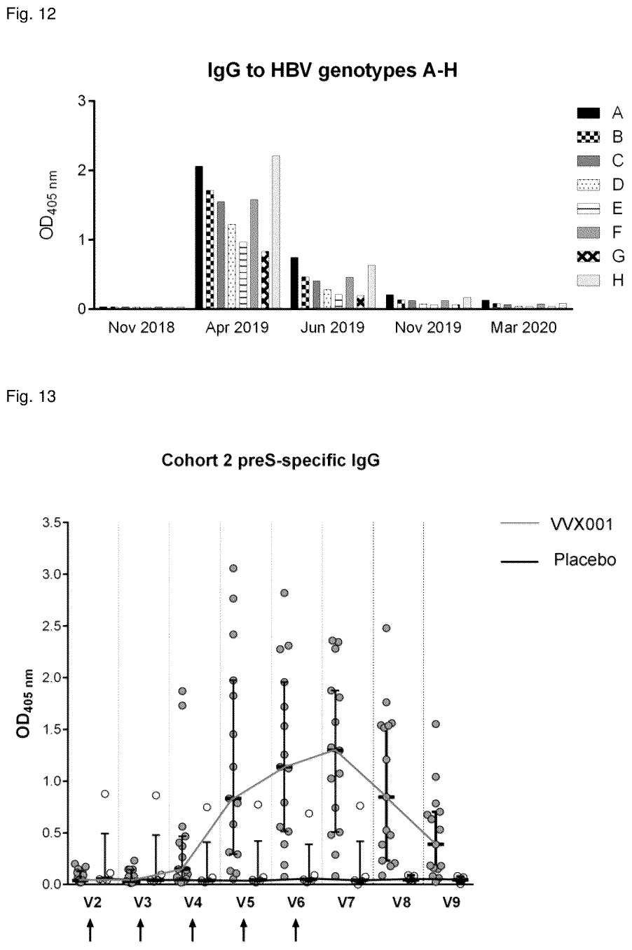

FIG. 11 shows the location of preS-derived peptides representing the different HBV genotypes which were used to investigate cross-reactivity of VVX001-induced preS-specific IgG with the HBV genotypes in FIG. 12.

FIG. 12 then demonstrates that the IgG antibodies induced by vaccination of the non-responder with VVX001 cross-reacted with the preS peptides from the HBV genotypes involved in binding to the NTCP receptor.

The finding that preS-specific IgG responses can be induced in a non-responder to HBV vaccines was confirmed in cohort 2 subjects. In fact, FIG. 13 shows that vaccination with VVX001 but not with placebo induced preS-specific IgG responses in non-responders and that HBV-specific immunological tolerance could be overcome by vaccination with VVX001.

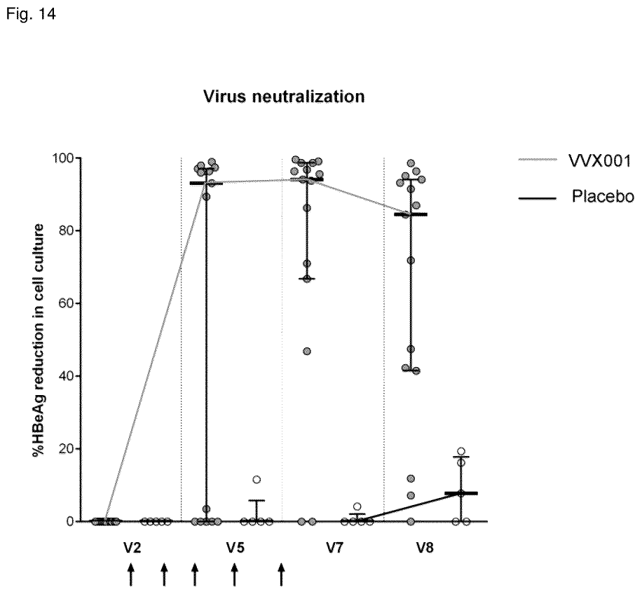

FIG. 14 shows that the preS-specific antibodies induced by vaccination with VVX001 in the HBV vaccine non-responders neutralized HBV infection.

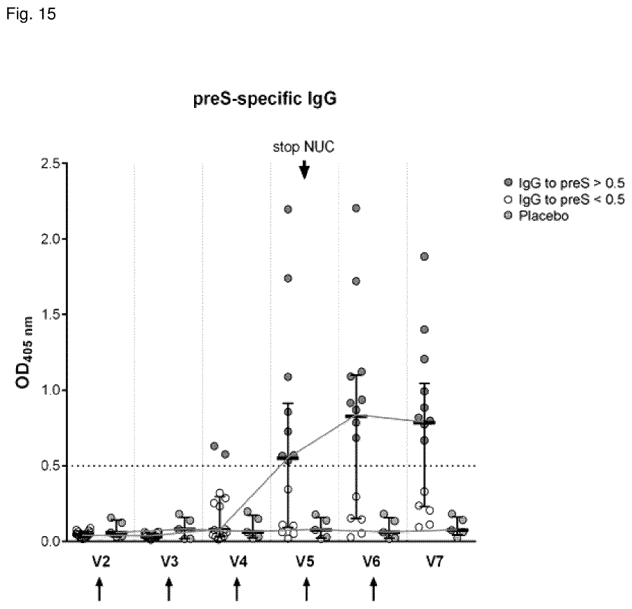

We were also able to show that vaccination with VVX001 could break immune tolerance in chronically infected HBV patients who had to be treated with NUC therapy. FIG. 15 shows that these cohort 4a patients did not mount preS-specific IgG production before vaccination with VVX001 (time point V2). Only VVX001 but not placebo-vaccinated cohort 4a patients produced then preS-specific IgG antibodies.

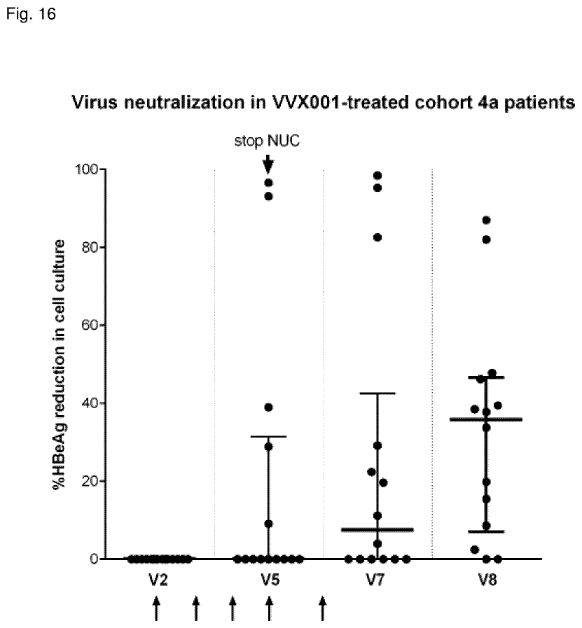

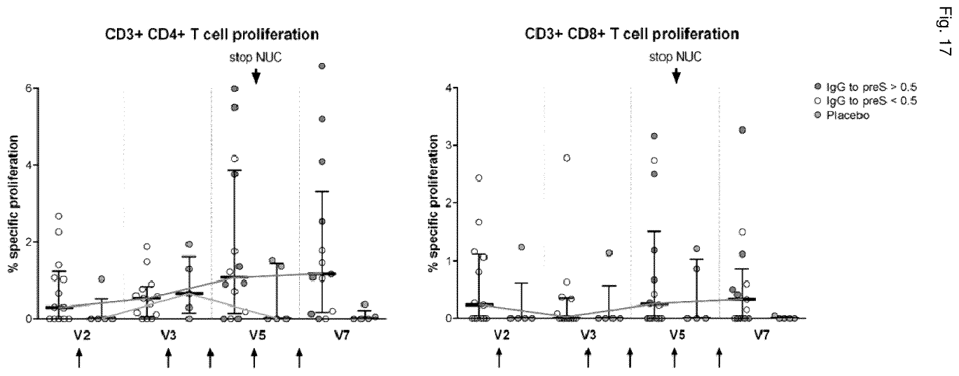

FIG. 16 shows that the preS-specific IgG antibodies induced by vaccination with VVX001 in cohort 4a patients enabled them to neutralize HBV infections.

Immunotolerance in cohort 4a by vaccination with VVX001 was not only overcome at the antibody but also at the T cell level.

FIG. 17 shows that vaccination with VVX001 but not with placebo induced preS-specific CD4+ (left) and CD8+ (right) T cell responses.

It has thus been demonstrated that vaccination with VVX001 induces preS-specific neutralizing antibodies and preS-specific T cell responses in subjects who did not respond to HBV vaccines and in chronically HBV-infected patients and thus could overcome HBV-specific immune tolerance.

### Materials and Methods

### Properties of the Vaccine, Immunization and Sampling Schedule

BM325=VVX001 is a recombinant fusion protein composed of preS from the large surface antigen of HBV and peptides derived from the grass pollen allergen Phl p 5. BM325 was produced by Biomay AG (Austria) according to GMP standards, formulated by adsorption to aluminum hydroxide as adjuvant (Zieglmayer et al., EBioMedicine 2016, 11:43-57). Alum-adsorbed BM325 (20 micrograms per injection) was applied subcutaneously.

### Determination of PreS-Specific Immune Response

The recombinant preS (preS1+preS2) from HBV genotype A2 (GenBank: AAT28735) and the preS-derived synthetic peptides spanning the whole preS sequence and representing the virus attachment site (FIG. 9) were produced as described (Tulaeva et al., EBioMedicine 2020;59:102953).

The specific IgG, IgG₁₋₄, IgM, IgA and IgE were measured by enzyme-linked immunosorbent assay (ELISA) according to the protocol described (Tulaeva et al., EBioMedicine 2020;59:102953).

To assess the specific T cell proliferation, freshly isolated peripheral blood mononuclear cells (PBMCs) were stained with carboxyfluorescein succinimidyl ester (CFSE) (Quah et al., Nature Protocols 2007, 2:2049-2056) and stimulated with 40 nm preS or preS-derived peptides as described (Cornelius et al., EBioMedicine. 2016; 11:58-67).

### Conclusion

VVX001 is a safe and well tolerated preS-based HBV vaccine and induces a protective preS-specific HBV-neutralizing immune response in subjects who are non-responders to HBV vaccines and in patients with chronic HBV infections. Vaccination with VVX001 overcomes HBV-specific immune tolerance in these subjects and hence can be used for preventive HBV vaccination in non-responders to S protein-based HBV vaccines and for therapeutic vaccination of chronically HBV-infected patients.

1. A method of inducing HBV neutralizing antibodies in a subject comprising administering to the subject a Hepatitis B virus (HBV) vaccine comprising a fusion protein of a preS polypeptide fused to at least one grass pollen allergen peptide, wherein the subject is an immune tolerant human subject and said treatment comprises repeated vaccination to break immune tolerance against HBV.

2. The method according to claim 1, wherein the immune tolerant subject is a human subject who is:
a) a patient with an acute or chronic HBV infection;
b) a patient who is chronically infected with HBV, wherein said patient is of an inactive carrier status; and/or
c) a low- or non-responder to a HBV vaccine, such as a HBsAg-based vaccine, or an S-protein-based HBV vaccine.

3. The method according to claim 1, wherein the subject is a HBsAg positive patient and/or at risk of suffering from HBV and/or Hepatitis D virus (HDV) disease or recurrent disease.

4. The method according to claim 1, wherein said administration is a prophylactic or therapeutic treatment.

5. The method according to claim 1, wherein said repeated vaccination comprises:
a) a basic immunization comprising at least 2, injections at intervals of 4-8 weeks, and
b) booster injections of at least 2, injections per year, following basic immunization.

6. The method according to claim 1, wherein the vaccine is administered at 10-100 μg preS per dose.

7. The method according to claim 1, wherein the preS polypeptide comprises at least 50% length of any one of SEQ ID NO: 1-8, and at least 80% sequence identity to the corresponding region of the respective SEQ ID NO: 1-8.

8. The method according to claim 1, wherein the grass pollen allergen peptide comprises any one of, or a fusion of more than one of SEQ ID NO:9-12.

9. The method according to claim 1, wherein the fusion protein is a single-chain fusion protein, wherein preS polypeptide and said at least one grass pollen allergen peptide are positioned in any order.

10. The method according to claim 1, wherein the fusion protein is formulated with an adjuvant, AS04, MF59, and toll-like receptor agonist adjuvants.

11. The method according to claim 1, wherein the subject is on standard antiviral treatment, or wherein the subject has discontinued NUC treatment.

12. The method according to claim 1, wherein upon administration, the subject comprises a anti-HBV immune response, in particular an anti-preS immune response.

13. The method according to claim 1, wherein the subject is a patient chronically infected with HBV who:
a) upon administration, mounts an immune response that neutralizes HBV; and/or
b) who is serum Hepatitis B S-antigen (HBSAg) positive.

14. (canceled)

15. (canceled)

16. The method of claim 5, wherein said basic immunization is up to 5 injections.

17. The method of claim 5, wherein said basic immunization is at monthly intervals.

18. The method of claim 5 wherein the booster injections are up to 4 injections per year following basic immunization.

19. The method of claim 9 wherein the fusion protein comprises an amino acid sequence having at least 80% sequence identity to SEQ ID NO:13 or SEQ ID NO: 14.

20. The method of claim 10, wherein the adjuvant is an alum.

21. The method of claim 20, wherein the alum is aluminum phosphate gel or aluminum hydroxide gel or mixture of the two.

22. The method of claim 10, wherein the toll-like receptor agonist adjuvants is a monophosphoryl lipid A and CpG.

23. The method of claim 11, wherein the standard antiviral treatment is nucleos(t)ide (NUC) treatment.

24. The method of claim 12, wherein the anti-HBV immune response is an IgG immune response.
